(19) United States
(12) Patent Application Publication
(10) Pub. No.: US 2021/0030994 A1
(43) Pub. Date: Feb. 4, 2021

(54) STRAP WITH ADHESIVE SILICONE ADJUSTMENT

(71) Applicant: KONINKLIJKE PHILIPS N.V., EINDHOVEN (NL)

(72) Inventor: Justin Edward Rothermel, Monroeville, PA (US)

(21) Appl. No.: 16/306,600

(22) PCT Filed: Jun. 28, 2017
(86) PCT No.: PCT/EP2017/066004

§ 371 Date: Dec. 3, 2018

Related U.S. Application Data
(60) Provisional application No. 62/355,377, filed on Jun. 28, 2016.

(51) Int. Cl. A61M 16/06; A62B 18/08; A62B 9/00; A62B 7/02

(52) U.S. Cl. CPC A61M 16/0688; A62B 7/02; A62B 9/00; A62B 18/084

(57) ABSTRACT

A strap member for securing a user interface device to the head of a user includes a first portion formed from a silicone or thermoplastic material; a second portion comprising an adhesive silicone material; and a third portion extending between the first and second portions. The second portion is positioned and structured to selectively adhere to the first portion.

### CROSS-REFERENCE TO RELATED APPLICATIONS

This patent application claims the priority benefit under 35 U.S.C. § 119(e) of U.S. Provisional Application No. 62/355,377 filed on Jun. 28, 2016, the contents of which are herein incorporated by reference.

### BACKGROUND OF THE INVENTION

### 1. Field of the Invention

The present invention pertains to strap members and retention mechanisms therefor, and more particularly to strap members formed from silicone or other similar materials and retention mechanisms therefor. The present invention also pertains to headgear assemblies including such a strap member. The present invention further pertains to an system for delivering a flow of breathing gas to the airway of a patient having such a headgear assembly including such a strap member.

### 2. Description of the Related Art

There are numerous situations where it is necessary or desirable to deliver a flow of breathing gas non-invasively to the airway of a patient, i.e., without intubating the patient or surgically inserting a tracheal tube in their esophagus. For example, it is known to ventilate a patient using a technique known as non-invasive ventilation. It is also known to deliver positive airway pressure (PAP) therapy to treat certain medical disorders, the most notable of which is OSA. Known PAP therapies include continuous positive airway pressure (CPAP), wherein a constant positive pressure is provided to the airway of the patient in order to splint open the patient's airway, and variable airway pressure, wherein the pressure provided to the airway of the patient is varied with the patient's respiratory cycle. Such therapies are typically provided to the patient at night while the patient is sleeping.

Non-invasive ventilation and pressure support therapies as just described involve the placement of a patient interface device including a mask component having a soft, flexible cushion on the face of a patient. The mask component may be, without limitation, a nasal mask that covers the patient's nose, a nasal cushion having nasal prongs that are received within the patient's nares, a nasal/oral mask that covers the patient's nose and mouth, or a full face mask that covers the patient's face. Such patient interface devices may also employ other patient contacting components, such as forehead supports, cheek pads and chin pads. The patient interface device is connected to a gas delivery tube or conduit and interfaces the ventilator or pressure support device with the airway of the patient, so that a flow of breathing gas can be delivered from the pressure/flow generating device to the airway of the patient. It is known to maintain such devices on the face of a wearer by a headgear having one or more straps adapted to fit over/around the patient's head. Because such respiratory patient interface devices are typically worn for an extended period of time, it is important for the headgear to maintain the mask component in a desired position while doing so in a manner that is comfortable to the patient.

Conventional headgear assemblies are commonly formed of panels and/or straps formed from fabric that is die-cut from generally flat sheet materials. The use of such fabric provides for the use of a “hook and loop” method of adjustment on some or all of the headgear straps. Without the use of rigid reinforcement in such headgear, the fabric panels often deform undesirably and result in buckling and mismatching when placed on a patient's head. Some of these issues can be self-compensating by stretching due to the elasticity of the materials used; however, such stretching may cause undesirable pressure points and unwanted tensions, thus causing discomfort to the patient.

One solution to making better fitting, and thus more comfortable, headgear has been to use straps formed in-whole or in-part from silicone which can readily be molded into three-dimensional shapes. However, the use of such straps typically requires the use of a thermoplastic clamp or clasp to retain the straps in a desired position. Such thermoplastic parts can be uncomfortable to a patient and can be generally costly to produce.

### SUMMARY OF THE INVENTION

Accordingly, as one aspect of the invention a strap member for securing a user interface device to the head of a user is provided. The strap member comprises: a first portion formed from a silicone or thermoplastic material; a second portion comprising an adhesive silicone material; and a third portion extending between the first and second portions, wherein the second portion is positioned and structured to selectively adhere to the first portion. The first portion may be formed from the silicone material and the third portion may be integrally formed from the silicone material. The adhesive silicone material may be in the form of a preformed pad and the third portion may be coupled to the preformed pad via overmolding. The second portion may be disposed as a coating on the third portion.

As another aspect of the invention, a headgear arrangement for securing a user interface device to the head of a user is provided. The headgear arrangement comprises a central portion having a plurality of strap members extending outward therefrom, each strap member comprising: a first portion formed from a silicone or thermoplastic material; a second portion comprising an adhesive silicone material; and a third portion extending between the first and second portions, wherein the second portion is positioned and structured to selectively adhere to the first portion.

As yet another aspect of the invention, a system for delivering a flow of breathing gas from a pressure generating device to the airway of a patient is provided. The system comprises: a delivery conduit having a first end structured to be coupled to the pressure generating device and a second end opposite the first end; a patient interface device coupled to the second end of the delivery conduit; and a headgear arrangement including a strap member. The strap member comprising: a first portion formed from a silicone or thermoplastic material; a second portion comprising an adhesive silicone material; and a third portion extending between the first and second portions, wherein the second portion is positioned and structured to selectively adhere to the first portion.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1 is a partially schematic view of a system adapted to provide a regiment of respiratory therapy to a user according to an exemplary embodiment of the invention;

FIG. 2 is a partially schematic view of a headgear arrangement according to an exemplary embodiment of the invention;

FIG. 3 is a detailed view of the portion of the headgear arrangement indicated at A in FIG. 2; and

FIG. 4 is a side edge view of a strap member and cooperating portion of a user interface device according to an exemplary embodiment of the invention.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

As used herein, the word “patient” or “user” shall be used interchangeably to refer to the person to which the interface device is delivering a flow of breathing gas. As used herein, the term “silicone material” shall be used to refer to a liquid silicone rubber (LSR) or RTV (room temperature vulcanization) rubbers or other soft materials that may be used as a headgear strap, such as a thermoplastic elastomer (TPE).

As used herein, the term “adhesive silicone material” shall be used to refer to a very low durometer silicone material which may be in an elastomeric or gel form and which provides a surface which is “sticky” or “tacky” to touch. Such materials are typically found in the Shore 00 durometer range, however even lower durometer variations may be employed which provide adhesion between two components.

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

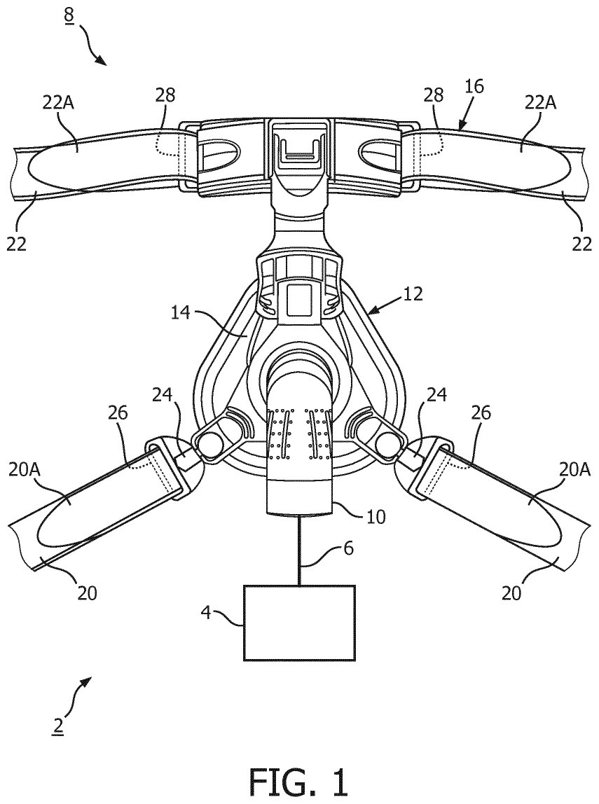

A system 2 adapted to provide a regimen of respiratory therapy to a patient according to one exemplary embodiment of the invention is generally shown in FIG. 1. System 2 includes a pressure generating device 4 (shown schematically), a delivery conduit 6 (shown partially schematically), and a patient interface device 8 having a fluid coupling conduit 10 coupled thereto. Pressure generating device 4 is structured to generate a flow of breathing gas and may include, without limitation, ventilators, constant pressure support devices (such as a continuous positive airway pressure device, or CPAP device), variable pressure devices (e.g., BiPAP®, Bi-Flex®, or C-Flex™ devices manufactured and distributed by Philips Respironics of Murrysville, Pa.), and auto-titration pressure support devices. Delivery conduit 6 is structured to communicate the flow of breathing gas from pressure generating device 4 to user interface device 8 through fluid coupling conduit 10, which in the illustrated embodiment is an elbow connector. Delivery conduit 6 and user interface device 8 are often collectively referred to as a user circuit.

A BiPAP® device is a bi-level device in which the pressure provided to the patient varies with the patient's respiratory cycle, so that a higher pressure is delivered during inspiration than during expiration. An auto-titration pressure support system is a system in which the pressure varies with the condition of the patient, such as whether the patient is snoring or experiencing an apnea or hypopnea. For present purposes, pressure/flow generating device 4 is also referred to as a gas flow generating device, because flow results when a pressure gradient is generated. The present invention contemplates that pressure/flow generating device 4 is any conventional system for delivering a flow of gas to an airway of a user of interface device 8 or for elevating a pressure of gas at an airway of the user, including the pressure support systems summarized above and non-invasive ventilation systems.

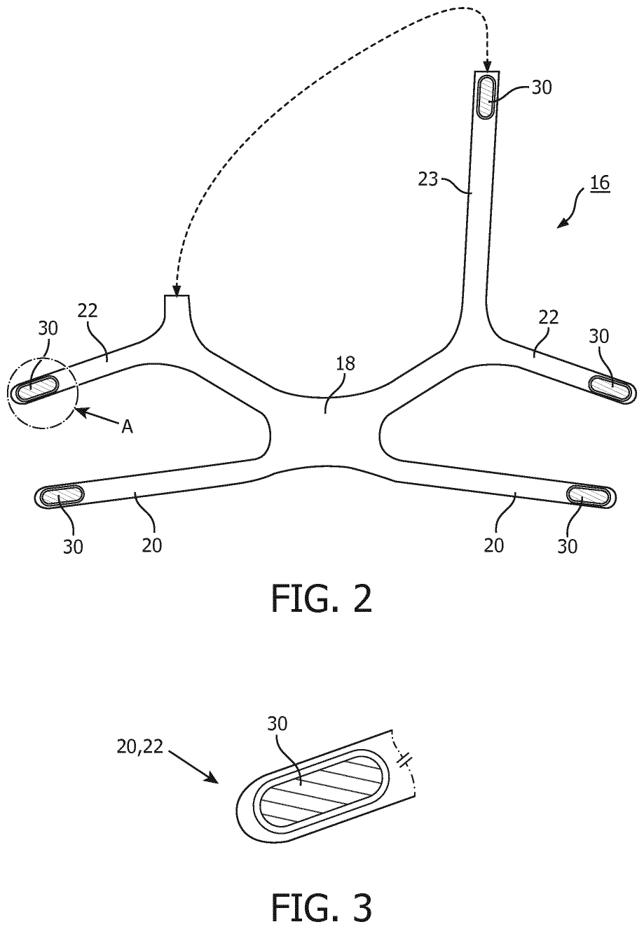

In the exemplary embodiment illustrated in FIG. 1, user interface device 8 is depicted as a nasal mask which includes a user sealing assembly or cushion 12 coupled to a generally rigid frame 14 which is coupled to conduit 6 via fluid coupling conduit 10. However, it is to be appreciated that other types of patient interface devices, such as, without limitation, a nasal pillows mask, an oral mask, a nasal/oral mask, or a full/total face mask (which encompasses the entire face), which facilitates the delivery of the flow of breathing gas to the airway of a user, may be substituted for user interface device 8 while remaining within the scope of the present invention. It is also to be appreciated that conduit 6 may be directly coupled to user interface device 8 without the use of any intermediary coupling, such as conduit 10. User interface device 8 may be secured to the head of a user via a suitable headgear 16, which is also shown in FIG. 2 for exemplary purposes only.

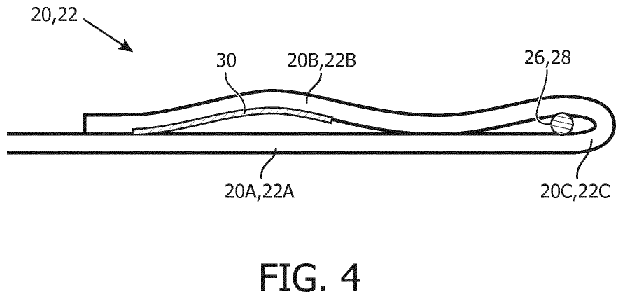

Continuing to refer to FIG. 1, as well as to FIG. 2, exemplary headgear 16 includes a central portion 18 which is positioned and structured to be disposed generally at the back of the head of a user. Headgear 16 further includes a number of lower strap members 20 and upper strap members 22 which each extend outward from central portion 18, as well as a top strap member 23 which generally extends from one of upper strap members 22. Each strap member 20, 22 is structured to selectively engage user interface device 8. As shown in the exemplary embodiment shown in FIG. 1, such engagement may be accomplished via a direct coupling of the strap portion to interface device 8 (such is the case with each of upper strap members 22) or via an indirect coupling by way of a suitable coupling element 24 (such is the case with each of lower strap members 20). In either case, such engagement is typically accomplished by folding the respective strap member 20, 22 around a portion of user interface device and back on itself, and then securing the folded-back portion to the remainder of the strap portion. An example of such arrangement is illustrated in FIG. 4 which shows how a second portion 20B, 22B of a strap member 20, 22 is folded around a corresponding portion 26, 28 of interface device 8 or coupling element 24 and back toward a first portion 20A, 22A, while a third portion 20C, 22C of each strap member 20, 22, which extends between first portions 20A, 22A and second portions 20B, 22B is in contact with portions 26, 28 of interface device 8.

In example embodiments of the present invention, headgear 16 and/or strap portions 20, 22 thereof, are formed in-whole or in-part from a silicone material. For example, a 20-30 Shore A durometer silicone may be molded in a more comfortable shape with varying wall thicknesses to improve the patient experience. Textures and surface treatments on the silicone may also be utilized help improve patient comfort and compliance. In order to provide for second portions 20B and 22B of strap members 20 and 22 to be secured to first portions 20A and 22A, respectively, after being folded around portions 26 and 28, each second portion 20B, 22B includes an adhesive silicone material 30 as previously described herein. Adhesive silicone material 30 may be in the form of a coating disposed on first portion 20B, 22B. Alternatively, adhesive silicone material 30 may be formed separately and then coupled to second portion 20B, 22B such as via over-molding or other suitable manufacturing process. In an example embodiment of the present invention, strap members having adhesive silicone pads formed from Silbione® Gel 4717 (manufactured by Bluestar Silicones) secured therein via over-molding have been successfully employed. The adhesive areas may be cleaned or renewed via suitable methods such that their performance can be sustained by the user. An additional benefit of such cleaning of both the adhesive as well as the strap portions is that there is very little to no drying time needed before use.

In another example embodiment of the present invention, a “peel and stick” method may be employed wherein the adhesive silicone material may serve as an intermediary component with adhesive surfaces on opposing sides.

In addition to being employed in adhering to strap surfaces formed from silicone, it is to be appreciated that the adhesive silicone arrangements described herein may also be employed to adhere to thermoplastic materials, such as are commonly presently employed as stiffening elements in fabric and silicone headgear arrangements.

It is also to be appreciated that although shown as generally single, oblong shaped areas, one or more the shape, size, or quantity of the adhesive portions may be varied without varying from the scope of the present invention. Such variations may be for functional, decorative, or a combination of such purposes.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment. Additionally, although the invention has been described in conjunction with a user interface device for delivering a flow of treatment gas to a user, it is to be appreciated that embodiments of the present invention may be utilized in other applications wherein strap members are employed without varying from the scope of the present invention.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

1. A strap member for securing a user interface device to the head of a user, the strap member comprising:
a first portion formed from a silicone or thermoplastic material;
a second portion comprising an adhesive silicone material; and
a third portion extending between the first and second portions,
wherein the second portion is positioned and structured to selectively adhere to the first portion.

2. The strap member of claim 1, wherein the first portion is formed from the silicone material and the third portion is integrally formed from the silicone material.

3. The strap member of claim 2, wherein the adhesive silicone material is in the form of a preformed pad and wherein the third portion is coupled to the preformed pad via overmolding.

4. The strap member of claim 2, wherein the second portion is disposed as a coating on the third portion.

5. A headgear arrangement for securing a user interface device to the head of a user, the headgear arrangement comprising a central portion having a plurality of strap members extending outward therefrom, each strap member comprising:
a first portion formed from a silicone or thermoplastic material;
a second portion comprising an adhesive silicone material; and
a third portion extending between the first and second portions, wherein the second portion is positioned and structured to selectively adhere to the first portion.

6. The headgear arrangement of claim 5, wherein the first portion of each strap member is formed from the silicone material and the third portion of each strap member is integrally formed from the silicone material.

7. The headgear arrangement claim 6, wherein the adhesive silicone material of each strap member is in the form of a preformed pad and wherein the third portion of each strap member is coupled to the respective preformed pad via overmolding.

8. The headgear arrangement of claim 6, wherein the second portion of each strap member is disposed as a coating on the third portion of each strap member.

9. A system for delivering a flow of breathing gas from a pressure generating device to the airway of a patient, the system comprising:
a delivery conduit having a first end structured to be coupled to the pressure generating device and a second end opposite the first end;
a patient interface device coupled to the second end of the delivery conduit; and
a headgear arrangement including a strap member comprising:
a first portion formed from a silicone or thermoplastic material;
a second portion comprising an adhesive silicone material; and
a third portion extending between the first and second portions, wherein the second portion is positioned and structured to selectively adhere to the first portion.

10. The system of claim 9, wherein the first portion is formed from the silicone material and the third portion is integrally formed from the silicone material.

11. The system of claim 10, wherein the adhesive silicone material is in the form of a preformed pad and wherein the third portion is coupled to the preformed pad via overmolding.

12. The system of claim 10, wherein the second portion is disposed as a coating on the third portion.
